(19) United States
(12) Patent Application Publication
(10) Pub. No.: US 2021/0260272 A1
(43) Pub. Date: Aug. 26, 2021

(54) METHOD AND CONTROL SYSTEM FOR A WOUND THERAPY APPARATUS

(71) Applicant: CONVATEC LIMITED, Flintshire (GB)

(72) Inventors: Ankur KODAVANTI, Banagalore (IN); Nandu NARAYANAN, Banagalore (IN); Praveen KADNI, Banagalore (IN); Srinivasan JAYARAMAN, Banagalore (IN); Sayantan B, Banagalore (IN)

(21) Appl. No.: 17/179,874

(22) Filed: Feb. 19, 2021

Related U.S. Application Data
(63) Continuation of application No. PCT/GB2021/050425, filed on Feb. 19, 2021.

(30) Foreign Application Priority Data
Feb. 20, 2020 (GB) 2002338.8

(51) Int. Cl. A61M 1/00; A61F 13/00

(52) U.S. Cl. CPC A61M 1/966; A61F 13/00068; A61M 2205/15; A61M 2205/581; A61M 2205/587; A61M 2205/583; A61M 2205/18; A61M 2205/3365

(57) ABSTRACT

The present invention relates to a method (10) and control system (100) for monitoring operation of a pressure gradient wound therapy apparatus (200). A parameter indicative of a rate of change of pressure within the applied wound dressing (202) is measured, and a rate of change of pressure within the applied wound dressing (202) in is determined in dependence on the measured parameter. The rate of change of pressure corresponds to a leak level and is used to control the wound therapy apparatus (200) in accordance with one or more predetermined actions such that an appropriate action is taken for a given leak level.

### CROSS-REFERENCE TO RELATED APPLICATIONS

This application is a continuation of International Application No. PCT/GB2021/050425 filed Feb. 19, 2021 and claims the priority of foreign Application No. GB2002338.8 filed Feb. 20, 2020. The disclosures of which are hereby incorporated herein in their entirety.

### TECHNICAL FIELD OF THE INVENTION

The present invention relates to a method and control system for a wound therapy apparatus. In particular, the invention relates to a method and control system for controlling operation of a wound therapy apparatus.

### BACKGROUND TO THE INVENTION

Pressure gradient wound therapy (positive or negative) is a known way of treating various wound types. Typically, this involves applying a pressure differential between a sealed region of a wound dressing and the surrounding environment to assist with healing the wound, e.g. through removal of oedema, increasing blood flow, mechanical contraction of the wound, increasing the formation of granulation tissue and/or active removal of excess exudate from the wound. Wound therapy of this type is particularly effective for the treatment of open traumatic, non-traumatic and chronic wounds. Such systems require a hermetic or near hermetic seal about the wound to perform adequately as any leak to or from the wound dressing makes it difficult to achieve the desired therapeutic pressure level within the wound dressing.

In practice, the seal about the wound is rarely completely hermetic, but in some instances such leaks may be acceptable. For instance, the level of leak may not be significant and a small reduction in the pressure differential between the wound dressing and the surrounding environment over time may have no or at least a minimal effect on the effectiveness of the therapy. In other cases the leak may be overcome through continual or intermittent operation of an associated pump assembly. Where the level of leak is significant the reduction in the pressure differential may not be manageable either because the leak level is too high to be counteracted by a pump, or where running a pump to compensate for the leak may not be practical—e.g. in terms of pump capability, energy consumption or excessive wear on the pump itself. In some instances, running the pump excessively may not be acceptable from a user experience point of view, either, for example on account of the noise. In such cases it may be appropriate to prevent further operation of the pump until the source of the leak has been identified and the problem rectified.

These issues are exacerbated in more modern, portable, disposable, pressure gradient wound therapy apparatus which tend to use smaller, less powerful pumps, which have a shorter lifespan and a limited power supply (e.g. batteries rather than mains power).

There is therefore a need to monitor (and if necessary, control) the operation of a wound therapy apparatus to ensure a desired pressure level is achievable within the wound dressing to aid healing of an associated wound.

It is an aim of an embodiment or embodiments of the invention to overcome or at least partially mitigate one or more problems with the prior art.

### SUMMARY OF THE INVENTION

According to an aspect of the invention there is provided a method for monitoring operation of a pressure gradient wound therapy apparatus, the method comprising: determining a leak level; categorising the leak level into one of a plurality of leak level categories; and controlling the wound therapy apparatus in accordance with one or more predetermined actions in dependence on the leak level category.

According to an aspect of the invention there is provided a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; categorising the leak level into one of a plurality of leak level categories; and controlling the wound therapy apparatus in accordance with one or more predetermined actions in dependence on the leak level category.

According to an aspect of the invention there is provided a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more predetermined actions in dependence on the leak level category.

The parameter indicative of the rate of change of pressure within the applied wound dressing may comprise a pressure value.

The method may comprise obtaining a first pressure value corresponding to the pressure within an applied wound dressing within a first time period. The method may comprise comparing the first pressure value with a second pressure value corresponding to the pressure within the applied wound dressing within a preceding time period. The comparison may be used to determine a rate of change of pressure within the applied wound dressing.

The method may comprise determining a rate of change of pressure within the applied wound dressing when a pump assembly of the wound therapy apparatus is deactivated. Advantageously, the method may be able to determine the presence and extent of a leak without that leak being masked by the operation of the pump assembly. The method may comprise determining a rate of change of pressure within the applied wound dressing when the pump assembly is active. Advantageously, knowledge of the speed at which the wound dressing may reach a desired pressure level may be indicative of the presence of and extent of a leak from the wound dressing. Further, some leaks may only be present whilst the pump assembly is active (e.g. leaks due to vibration of the wound therapy apparatus caused by the pump assembly being active), and hence may only be identified by determining the rate of change of pressure within the applied wound dressing when the pump assembly is active.

In embodiments, the method may comprise determining a first rate of change of pressure within the applied wound dressing when the pump assembly is active, and determining a second rate of change of pressure within the applied wound dressing when the pump assembly is deactivated. The method may comprise comparing the first rate of change of pressure with the second rate of change of pressure. The method may comprise determining a leak rate from the wound dressing in dependence on said comparison.

The parameter indicative of the rate of change of pressure within the applied wound dressing may comprise, or may be calculated from, a measured time value. The time value may correspond to the time between successive operations of a component of the wound therapy apparatus. For example, the time value may correspond to the time between successive operating cycles of a pump assembly of the wound therapy apparatus. In such embodiments, the pump assembly of the apparatus may be configured to activate upon the pressure level inside the wound dressing reaching a first threshold level (e.g. an activation threshold level) and deactivate upon the pressure level inside the wound dressing reaching a second threshold level (e.g. a deactivation threshold level).

Thus one embodiment provides a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more predetermined actions in dependence on the leak level category; wherein the parameter indicative of the rate of change of pressure within the applied wound dressing comprises, or is calculated from, a time value; and wherein the time value corresponds to the time between successive operating cycles of a pump assembly of the wound therapy apparatus. In embodiments, the method may comprise measuring a time value corresponding to the time between the end of a first operating cycle of the pump assembly (e.g. upon deactivation of the pump assembly) and the start of a second operating cycle of the pump assembly (e.g. upon subsequent activation of the pump assembly). In such embodiments, the parameter comprises the time taken for the pressure level within the wound dressing to change (e.g. drop) from the second threshold level to the first threshold level when the pump assembly is deactivated.

In some embodiments, the method may comprise comparing the time value, calculated by measuring the time between the end of a first operating cycle of the pump assembly and the start of a second operating cycle of the pump assembly, with a second time value corresponding to the length of time of an operating cycle (e.g. the second operating cycle) of the pump assembly. In such embodiments, the method may comprise determining a parameter in the form of a ratio of the time for which the pump assembly is not operating and the time for which the pump assembly is operating. The ratio may be determined as a percentage of time for which the pump assembly is not operating during a single duty cycle of the pump assembly.

Thus, one embodiment provides a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more predetermined actions in dependence on the leak level category; the method including the steps of measuring a time value corresponding to the time between the end of a first operating cycle of the pump assembly and the start of a second operating cycle of the pump assembly; and comparing the time value with a second time value corresponding to the length of time of an operating cycle of the pump assembly to determine a ratio of the time for which the pump assembly is not operating and the time for which the pump assembly is operating.

The parameter indicative of the rate of change of pressure within the applied wound dressing may comprise a parameter indicative of an energy consumption of a power source associated with the wound therapy apparatus. For example, the parameter may comprise a parameter indicative of an energy consumption of a power source which is configured to power a pump assembly of the wound therapy apparatus, in use. The power consumed by the pump assembly may be indicative of a leak level from the applied wound dressing insofar as the pump assembly may be required to be active for longer, and/or more frequently in the presence of a leak from the wound dressing in order to maintain a target pressure within the dressing. In embodiments, the parameter comprises a parameter indicative of a voltage level associated with the power source. In some embodiments, the parameter comprises a parameter indicative of a drain voltage of the power source during operation of a pump assembly of the wound therapy apparatus.

According to an aspect of the invention there is provided a method for monitoring operation of a pressure gradient wound therapy apparatus, the method comprising: obtaining a first pressure value corresponding to the pressure within an applied wound dressing within a first time period; comparing the first pressure value with a second pressure value corresponding to the pressure within the applied wound dressing within a preceding time period to determine a rate of change of pressure within the applied wound dressing, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more predetermined actions in dependence on the leak level category.

Advantageously, the method accounts for a plurality of different leak levels from a wound dressing. In some instances, certain actions may not be relevant or indeed suitable for certain leak levels. For example, certain leak levels which are deemed acceptable may be managed by increasing or moderating operation of a pump assembly of the wound therapy apparatus in order to maintain a desired pressure level within the wound dressing. However, where a major leak is identified e.g. which may be deemed unacceptable, such a response may not be suitable as this may cause excessive energy consumption, noise or wear on the apparatus itself. In such instances, it may be beneficial to shut down the apparatus (at least partially—for example shutting off the pump, whilst operating, e.g. flashing, a warning light) until the leak can be addressed. For lower leak levels, the drop off in pressure (negative or positive) within the dressing may be acceptable without necessitating control of the pump assembly. Accordingly, an indication to a user may be sufficient here, for example, to prompt the user to check the seal of the wound dressing. By categorising the leak level into a plurality of categories and controlling the wound therapy apparatus in accordance with that classification, the method of the present invention advantageously performs appropriate action in a number of different scenarios.

When referring to a “higher” leak level category this should be interpreted as a category corresponding to a larger leak from the wound dressing. For negative pressure wound therapy this would correspond to a category where the pressure inside the wound dressing is increasing at a greater rate for “higher” leak levels. In contrast, for positive pressure wound therapy, this would correspond to a category wherein the pressure inside the wound dressing is decreasing at a greater rate for “higher” leak levels. It follows that a “lower” leak level category should be interpreted as a category corresponding to a smaller leak from the wound dressing—i.e. a slower rate of change of pressure within the wound dressing.

When used herein and throughout the specification the term “pressure gradient wound therapy apparatus” is intended to cover a wound therapy apparatus wherein a pressure differential (either positive or negative) is applied between a sealed region of the wound dressing and the surrounding environment. In embodiments, the method comprises monitoring operation of a negative pressure wound therapy apparatus. In other embodiments, the method comprises monitoring operation of a positive pressure wound therapy apparatus.

As used herein, negative pressure wound therapy is a therapeutic technique using a suction dressing to remove excess exudation and promote healing in acute or chronic wounds. A vacuum of −50 to −200 mm Hg, or −75 to −150 mm Hg may be applied with typical negative pressure of −80 to −130 mm Hg, −100 to −130 mm Hg, or often about −125 mm Hg being applied to a wound.

For positive pressure wound therapy, a net positive pressure is applied to the wound, which may include providing simultaneous aspiration and irrigation of the wound. Positive pressure wound therapy may be carried out at a positive pressure of up to 50% atm., typically at a low positive pressure of up to 20% atm., more usually up to 10% atm. at the wound. Positive pressure wound therapy is known and referred to in US20180140755.

Optional features set out below may apply to any aspect of the invention.

In embodiments the method comprises categorising the leak level into leak level categories corresponding to one or more of: a no leak category; a minor leak category; and a major leak category. One or more further leak level categories may be used, such as a medium or intermediate leak level category between a minor leak category and a major leak category.

A no leak category may correspond to a leak level where there is substantially no change in pressure within the applied wound dressing—i.e. a situation where there is a complete hermetic seal about the wound dressing. A no leak category may also correspond to an “acceptable” leak level. For example, an acceptable leak level may be defined as a leak level where the change in pressure as a result of a leak is acceptable or at least manageable through operation of the wound therapy apparatus—e.g. through intermittent activation of the pump assembly without requiring the user to take any action to address the leak specifically.

The leak levels associated with each leak level category may be predetermined. In other embodiments, the leak levels for each category may be dependent on one or more factors, and may optionally be configurable, in use. The leak levels corresponding to each leak level category may be defined in dependence on a capability (e.g. an air flow capability) of a pump assembly of the apparatus. For instance, the capability of the pump assembly may affect the time taken to reach a desired pressure level within the wound dressing, it may affect the noise output from the apparatus in achieving a desired pressure level, it may even affect the ability of the pump assembly to overcome the leak altogether. Accordingly, a pump assembly with a greater capability compared with one with a lower capability may be able to overcome higher leak levels more quickly and efficiently. In embodiments the leak levels corresponding to each leak level category may be defined in dependence on an energy capacity of a power source for the apparatus.

A no leak level may correspond to a leak rate (i.e. a change in pressure) of up to 0.50 mmHg/s, or up to 0.45 mmHg/s, or up to 0.40 mmHg/s, or up to 0.30 mmHg/s, or up to 0.20 mmHg/s, for example. In presently preferred embodiments a no leak level corresponds to a leak rate between 0-0.50 mmHg/s.

A minor leak level may correspond to a leak rate of up to 1.00 mmHg/s, or up to 0.80 mmHg/s, or up to 0.60 mmHg/s, or up to 0.40 mmHg/s, for example. A minor leak level may correspond to a leak rate of no more than 2.00 mmHg/s, or no more than 1.50 mmHg/s, or no more than 1.00 mmHg/s, or no more than 0.50 mmHg/s, for example. In embodiments, a minor leak level may be defined as having a leak rate between 0.20-1.00 mmHg/s, or between 0.30-0.70 mmHg/s, for example. In presently preferred embodiments a minor leak level corresponds to a leak rate between 0.50-1.00 mmHg/s.

A medium or intermediate leak level may correspond to a leak rate of up to 4.00 mmHg/s, or up to 3.00 mmHg/s, or up to 2.00 mmHg/s, or up to 1.00 mmHg/s, or up to 0.75 mmHg/s, or up to 0.5 mmHg/s, for example. A medium or intermediate leak level may correspond to a leak rate of no more than 4.00 mmHg/s, or no more than 3.00 mmHg/s, or no more than 2.00 mmHg/s, or no more than 1.00 mmHg/s, for example. In embodiments, a medium or intermediate leak level may be defined as having a leak rate between 0.30-4.00 mmHg/s, or between 0.70-2.00 mmHg/s, for example. In presently preferred embodiments a medium or intermediate leak level corresponds to a leak rate between 1.00-2.00 mmHg/s.

A high leak level may correspond to a leak rate of more than 1.00 mmHg/s, or more than 2.00 mmHg/s, or more than 3.00 mmHg/s, or more than 4.00 mmHg/s, for example. In presently preferred embodiments a high leak level corresponds to a leak rate of more than 2.00 mmHg/s.

In embodiments, a no leak level may correspond the pump assembly not operating for at least 75%, or at least 80%, or at least 85%, or at least 90%, or at least 95% of a duty cycle of the pump assembly. In presently preferred embodiments a no leak level corresponds to the pump assembly not operating for at least 80% of a duty cycle of the pump assembly.

A minor leak level may correspond to the pump assembly not operating for at least 60%, or at least 65%, or at least 70%, or at least 75%, or at least 80% of a duty cycle of the pump assembly. A minor leak level may correspond to the pump assembly not operating for up to 85%, or up to 80%, or up to 75%, or up to 70%, or up to 65% of a duty cycle of the pump assembly. In embodiments, a minor leak level may correspond to the pump assembly not operating for between 60-85%, or between 70-80%, or between 75-85% of a duty cycle of the pump assembly. In presently preferred embodiments a minor leak level corresponds to the pump assembly not operating for between 70-80% of a duty cycle of the pump assembly.

A medium or intermediate leak level may correspond to the pump assembly not operating for at least 25%, or at least 30%, or at least 40%, or at least 50%, or at least 60%, or at least 70%, or at least 75% of a duty cycle of the pump assembly. A medium or intermediate leak level may correspond to the pump assembly not operating for up to 80%, or up to 75%, or up to 70%, or up to 60%, or up to 50%, or up to 40%, or up to 30% of a duty cycle of the pump assembly. In embodiments, a medium or intermediate leak level may correspond to the pump assembly not operating for between 30-80%, or between 40-70%, or between 50-60% of a duty cycle of the pump assembly. In presently preferred embodiments a medium or intermediate leak level corresponds to the pump assembly not operating for between 30-70% of a duty cycle of the pump assembly.

A major leak level may correspond to the pump assembly not operating for no more than 10%, or no more than 20%, or no more than 30%, or no more than 40%, or no more than 50% of a duty cycle of the pump assembly. In presently preferred embodiments, a major leak level corresponds to the pump assembly not operating for no more than 30% of a duty cycle of the pump assembly.

In embodiments wherein the method comprises obtaining a pressure value, e.g. the first and second pressure values, the or each pressure value may correspond to an absolute pressure value within the applied wound dressing. In some embodiments, the or each pressure value may correspond to a relative pressure value within the applied wound dressing, which may be relative to atmospheric pressure or to a desired/optimum pressure value for the wound dressing.

The method may comprise controlling an operating level of a component of the wound therapy apparatus in dependence on the leak level category. The component may comprise a pump assembly of the wound therapy apparatus. In such embodiments, the operating level may comprise a power output or motor speed of the pump assembly, for example. In some embodiments the method may comprise activating the pump assembly in response to an increase in the leak level—e.g. a change in a leak level such that it is categorised in a leak level category corresponding to a higher leak level. The method may comprise increasing the power output/motor speed of the pump assembly in response to an increase in the leak level. Advantageously, the pump assembly may be controlled to overcome the identified leak and thereby reach a desired pressure level within the wound dressing. In further embodiments, the method may comprise disabling operation of the pump assembly in response to an increase in the leak level. For instance, in some cases the leak level may be such that the pump assembly cannot act to reach the desired pressure level within the wound dressing, or in doing so would result in excessive energy consumption or wear on components of the pump assembly. Accordingly, it may be advantageous to cease operation of the pump assembly to prevent excess energy consumption and/or damage to the pump assembly.

The method may comprise controlling the duration for which the pump assembly can operate for in dependence on the leak level category. The duration may correspond to an absolute duration—e.g. a set length of time measured in seconds, minutes, etc. —or may correspond to a number of operating or duty cycles of the pump assembly—e.g. a set number of operating or duty cycles. For example, the method may comprise operating (or allowing operation of) the pump assembly for different durations in dependence on the leak level category. The method may comprise allowing the pump assembly to operate for a longer duration in dependence on a determination of a leak in a lower leak level category when compared with a leak in a higher leak level category. Advantageously, the method may comprise controlling the duration for which the pump assembly can operate for in a given leak state to prevent excessive use and wear of the pump assembly which may otherwise be present if the pump assembly is allowed to operate for an extended duration when a high or major leak is present. The method may comprise periodically determining a leak level category during operation of the pump to determine a change in leak level category. The method may comprise adjusting the duration for which the pump assembly can operate in dependence on change in leak level category.

The method may comprise outputting an indication to a user of the apparatus in dependence on the leak level category. The indication may include one or more of illuminating a light, controlling an associated display, and/or activating an alert to inform the user of the apparatus of the presence of a leak. The indication may comprise information indicative of the leak level category. For example, the indication may inform the user of the leak level category directly. The method may comprise outputting instructions for a user to address the leak in dependence on the leak level category—e.g. increase pump output, deactivate pump, re-seal/replace wound dressing. The user may then act in response to such an output. In some embodiments the indication may be purely informative and be indicative of one or more actions taken in dependence on the leak level category, for instance in embodiments wherein the method comprises automatically controlling operation of the pump assembly in dependence on the leak level category.

In embodiments, the method may comprise illuminating a light at different frequencies in dependence on the leak level category. For example, the method may comprise illuminating, or “flashing” the light at a first frequency in dependence on a determination of a leak in a first leak level category, and illuminating/flashing the light at a second frequency in dependence on a determination of a leak in a second leak level category. The method may comprise illuminating/flashing the light at a faster frequency for a higher leak level category when compared with a lower leak level category. The method may comprise illuminating/flashing the light as a frequency of 1, or up to 2, or up to 3, or up to 4, or up to 5, or more than 5 times per second.

The method may comprise illuminating a light indicative of the operational state of one or more components of the wound therapy apparatus, e.g. the pump assembly. For example, the method may comprise illuminating a light to indicate that the pump assembly is active. This may include illuminating a light to indicate that the pump assembly is actively increasing or reducing the pressure within the applied wound dressing, or is able to (e.g. it has not been disabled) control the pressure level within the applied wound dressing. The method may comprise switching the light off in dependence on the leak level category, or in dependence on the number of operational cycles of the pump assembly. The method may comprise illuminating a light indicative of the pressure level within the applied wound dressing. For example, in some embodiments the method may comprise illuminating a light to indicate that a target pressure level has been reached within the applied wound dressing.

The method may comprise controlling the brightness of a light in dependence on the leak level category. For example, the method may comprise controlling (e.g. via phase width modulation) the brightness of the light, or a sequenced change in the brightness of the light in dependence on the leak level category. The method may comprise illuminating the light at a first brightness, or in a first illumination sequence, in dependence on a determination of a leak in a first leak level category, and illuminating at a second brightness, or in a second illumination sequence in dependence on a determination of a leak in a second leak level category.

The method may comprise controlling the colour of a light in dependence on the leak level category. For example, the method may comprise providing an indication in the form of a light of a first colour in dependence on a determination of a leak in a first leak level category, and providing an indication in the form of the light of a second colour dependence on a determination of a leak in a second leak level category.

In embodiments, the method may comprise illuminating one or more of a plurality of lights in dependence on the determined leak level category. For example, in some embodiments the method may comprise illuminating a first combination of one or more of the plurality of lights in dependence on a determination of a leak in a first leak level category, and illuminating a second combination of one or more of the plurality of lights in dependence on a determination of a leak in a second leak level category. The method may comprise illuminating a first light to indicate the determined leak level, and a second light to indicate that the pump assembly of the wound therapy apparatus is active. The method may comprise illuminating the first light but deactivating the second light in dependence on the leak level category, or in dependence on a number of (optionally failed) attempts to achieve a target pressure level within the applied wound dressing. Accordingly, the method may comprise indicating to a user that the pump assembly has been unable to obtain a target pressure level within the applied wound dressing.

In embodiments, the method comprises controlling the wound therapy apparatus in accordance with one or more different predetermined actions in dependence on the leak level category. For example, in some embodiments the method comprises: controlling the wound therapy apparatus in accordance with a first set of instructions in dependence on a determination of a leak level in a first leak level category; and controlling the wound therapy apparatus in accordance with a second set of instructions in dependence on a determination of a leak level in a second leak level category. The method may extend to controlling the wound therapy apparatus in accordance with a third set of instructions in dependence on a determination of a leak level in a third leak level category. The method may extend to controlling the wound therapy apparatus in accordance with a fourth set of instructions in dependence on a determination of a leak level in a fourth leak level category For instance, in embodiments the method comprises: taking no action in dependence on a determination of a leak level in a no leak category; outputting an indication to a user of the apparatus in dependence on a determination of a leak level in a minor leak level category; controlling an operating level of a pump assembly of the wound therapy apparatus in dependence on a determination of a leak level in a medium leak level category; and preventing operation of the pump assembly in dependence on a determination of a leak level in a major leak level category.

Thus, one embodiment of the invention provides a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more different instructions for predetermined actions in dependence on the leak level category; controlling the wound therapy apparatus in accordance with a first set of instructions in dependence on a determination of a leak level in a first leak level category; controlling the wound therapy apparatus in accordance with a second set of instructions in dependence on a determination of a leak level in a second leak level category; and controlling the wound therapy apparatus in accordance with a third set of instructions in dependence on a determination of a leak level in a third leak level category.

Similarly, one embodiment of the invention provides a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more predetermined actions in dependence on the leak level category as follows: taking no action in dependence on a determination of a leak level in a no leak category; outputting an indication to a user of the apparatus in dependence on a determination of a leak level in a minor leak level category; controlling an operating level of a pump assembly of the wound therapy apparatus in dependence on a determination of a leak level in a medium leak level category; and preventing operation of the pump assembly in dependence on a determination of a leak level in a major leak level category.

The method may comprise controlling the wound therapy apparatus in accordance with a combination of different predetermined actions in dependence on the leak level category. For example, for some leak level categories, the method may comprise both controlling an operating level of a pump assembly of the wound therapy apparatus and outputting an indication to the user indicative of the leak level and/or the change in operating level of the pump assembly. For instance, in embodiments the method comprises: taking no action in dependence on a determination of a leak level in a no leak category; controlling an operating level of a pump assembly of the wound apparatus and outputting a first indication to a user of the apparatus in dependence on a determination of a leak level in a minor leak level category; controlling an operating level of a pump assembly of the wound therapy apparatus and outputting a second indication to a user of the apparatus in dependence on a determination of a leak level in a medium leak level category; and preventing operation of the pump assembly and outputting a third indication to a user of the apparatus in dependence on a determination of a leak level in a major leak level category. The first, second and/or third indications may be different. For instance, for a low level the indication may comprise a visual indication, only. However, for higher leak level categories the indication may comprise both a visual and audible indication such as an alert or alarm.

The method may comprise obtaining a plurality of pressure values within the first time period. In such embodiments, the method may comprise obtaining an average of the plurality of pressure values obtained within the first time period to obtain the first pressure value. Similarly, the method may comprise obtaining a plurality of pressure values within the preceding time period. In such embodiments, the method may comprise obtaining an average of the plurality of pressure values obtained within the preceding time period to obtain the second pressure value.

The first time period may comprise a time period of at least 10 ms, or at least 20 ms, or at least 50 ms, or at least 100 ms, or at least 150 ms, for example. The first time period may comprise a time period of no more than 500 ms, or no more than 250 ms, or no more than 200 ms, or no more than 150 ms, or no more than 100 ms, or no more than 50 ms, for example. In some embodiments the first time period may comprise a time period of approximately 200 ms.

The method may comprise obtaining a plurality of pressure values at intervals within the first time period. For example, the method may comprise obtaining pressure values at intervals of approximately 10 ms. The method may comprise obtaining at least 1, or at least 2, or at least 5, or at least 10, or at least 15, or at least 20, or at least 50 pressure values within the first time period.

The preceding time period may comprise a time period of at least 10 ms, or at least 20 ms, or at least 50 ms, or at least 100 ms, or at least 150 ms, for example. The preceding time period may comprise a time period of no more than 500 ms, or no more than 250 ms, or no more than 200 ms, or no more than 150 ms, or no more than 100 ms, or no more than 50 ms, for example. In some embodiments the preceding time period may comprise a time period of approximately 200 ms. In embodiments, the preceding time period is equal in length to the first time period.

The method may comprise obtaining a plurality of pressure values at intervals within the preceding time period. For example, the method may comprise obtaining pressure values at intervals of approximately 10 ms. The method may comprise obtaining at least 1, or at least 2, or at least 5, or at least 10, or at least 15, or at least 20, or at least 50 pressure values within the preceding time period.

The method may comprise comparing the rate of change of pressure to a first leak threshold and categorising the corresponding leak level in a first leak level category if the rate of change of pressure is below the first leak threshold. The method may comprise categorising the corresponding leak level in a second leak level category if the rate of change of pressure is above the first leak threshold. The method may comprise comparing the rate of change of pressure to a second leak threshold and categorising the corresponding leak level in a second leak level category if the rate of change of pressure is below the second leak threshold but above the first leak threshold. The invention may extend to comparing the rate of change of pressure to a third leak threshold and categorising the corresponding leak level in a third leak level category if the rate of change of pressure is below the third leak threshold but above the second leak threshold. The method may comprise categorising the corresponding leak level in a fourth leak level category if the rate of change of pressure is above the third leak threshold. The first leak level category may be a no leak category. The second leak level category may be a minor leak level category. The third leak level category may be a medium leak level category. The fourth leak level category may be a major leak level category.

The first leak threshold may correspond to a leak rate (e.g. a rate of change of pressure) of up to 1.00 mmHg/s, or up to 0.75 mmHg/s, or up to 0.50 mmHg/s, or up to 0.40 mmHg/s, or up to 0.30 mmHg/s, or up to 0.20 mmHg/s, for example. In presently preferred embodiments the first leak threshold corresponds to a leak rate of approximately 0.50 mmHg/s. The second leak threshold may correspond to a leak rate of up to 2.00 mmHg/s, or up to 1.50 mmHg/s, or up to 1.25 mmHg/s, or up to 1.00 mmHg/s, or up to 0.80 mmHg/s, or up to 0.60 mmHg/s, or up to 0.40 mmHg/s, for example. In presently preferred embodiments the second leak threshold corresponds to a leak rate of approximately 1.00 mmHg/s. The third leak threshold may correspond to a leak rate of up to 4.00 mmHg/s, or up to 3.00 mmHg/s, or up to 2.00 mmHg/s, or up to 1.00 mmHg/s, or up to 0.75 mmHg/s, or up to 0.5 mmHg/s, for example. In presently preferred embodiments the third leak threshold corresponds to a leak rate of approximately 2.00 mmHg/s.

In embodiments, the method comprises comparing the determined rate of change of pressure (e.g. the rate of change of pressure corresponding to the first pressure value) with a rate of change of pressure observed at one or more preceding time periods. For example, the method may comprise obtaining an average rate of change of pressure observed at a plurality of preceding time periods, and comparing the determined rate of change of pressure with the average rate of change of pressure observed at the plurality of preceding time periods. The method may comprise identifying the presence of a leak and/or determining a leak level in dependence on this comparison. Advantageously, the method may be able to identify a change in the leak level of the wound dressing if the determined rate of change of pressure differs significantly from those observed at the one or more preceding time periods.

Thus one embodiment of the invention provides a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; comparing the determined rate of change of pressure to a plurality of leak thresholds to categorise the leak level into one of a plurality of leak level categories; and controlling the wound therapy apparatus in accordance with one or more predetermined actions in dependence on the leak level category; wherein the determined rate of change of pressure is compared with a rate of change of pressure observed at one or more preceding time periods.

Similarly, one embodiment of the invention provides a method for monitoring operation of a pressure gradient wound therapy apparatus, the method comprising: measuring a parameter indicative of a rate of change of pressure within the applied wound dressing; determining a rate of change of pressure within the applied wound dressing in dependence on the measured parameter, the rate of change of pressure corresponding to a leak level; obtaining an average rate of change of pressure observed at a plurality of preceding time periods; and comparing the determined rate of change of pressure to a plurality of leak thresholds to categorise the leak level into one of a plurality of leak level categories; and controlling the wound therapy apparatus in accordance with one or more predetermined actions in dependence on the leak level category; wherein the determined rate of change of pressure is compared with the average rate of change of pressure observed at the plurality of preceding time periods.

The method may comprise determining a rate of change of pressure within the wound dressing as follows:

$$Rate of change of pressure = \vert \frac{{P}_{m} - {P}_{m - 1}}{{T}_{m} - {T}_{m - 1}} \vert \qquad [ Equation 1 ]$$

where Pₘ is the first pressure value, Pₘ₋₁ is the second pressure value, Tₘ corresponds to a time stamp of the first time period, and Tₘ₋₁ corresponds to a time stamp of the preceding time period.

The method may be performed only when a pump assembly of the wound therapy apparatus is inactive—i.e. when it is not providing a source of positive or negative pressure to the wound dressing. In this way, the method may be able to identify leaks within the wound dressing which may otherwise be masked by operation of the pump assembly.

The method may comprise obtaining a pressure value, e.g. the first and/or second pressure values using one or more sensors (e.g. pressure sensors) associated with the wound dressing. The one or more sensors may be embedded within the wound dressing, and/or may be arranged about a periphery of the wound dressing. In embodiments, the one or more sensors may comprise micro-electromechanical system (MEMS) or nano-electromechanical system (NEMS) sensors. The one or more sensors may be arranged as a strip of sensors about a periphery of a wound dressing. The one or more sensors may be associated with a pump assembly of the wound therapy apparatus. The one or more sensors may be associated with another part of the wound therapy apparatus. For example, the one or more sensors may be located in a housing with the pump, and/or arranged between a pump and a wound dressing of the wound therapy apparatus, e.g. associated with a conduit or tube connecting the wound dressing and the pump, or associated with a canister forming part of the wound therapy apparatus.

According to an aspect of the invention there is provided a control system for monitoring operation of a pressure gradient wound therapy apparatus, the control system comprising one or more controllers, the control system being configured to carry out any one of the methods of the above aspects of the invention, optionally including any optional feature, and output a control signal to control the wound therapy apparatus in accordance with one or more predetermined actions in dependence on the leak level category.

According to an aspect of the invention there is provided a control system for monitoring operation of a pressure gradient wound therapy apparatus, the control system comprising one or more controllers, the control system being configured to: receive an input signal indicative of a parameter indicative of a rate of change of pressure within the applied wound dressing; determine a rate of change of pressure within the applied wound dressing in dependence on the parameter, the rate of change of pressure corresponding to a leak level; compare the determined rate of change of pressure to a plurality of leak thresholds to categorise the leak level into one of a plurality of leak level categories; and output a control signal to control the wound therapy apparatus in accordance with one or more predetermined actions in dependence on the leak level category.

The parameter indicative of the rate of change of pressure within the applied wound dressing may comprise a pressure value.

The control system may be configured to receive an input signal indicative of the first pressure value. The first pressure value may correspond to the pressure within an applied wound dressing within a first time period. The control system may be configured to compare the first pressure value with a second pressure value corresponding to the pressure within the applied wound dressing within a preceding time period. The control system may be configured to perform this comparison to determine a rate of change of pressure within the applied wound dressing.

The parameter indicative of the rate of change of pressure within the applied wound dressing may comprise, or may be calculated from, a time value. In such embodiments, the control system may be configured to receive an input signal indicative of a time value. The time value may correspond to the time between successive operations of a component of the wound therapy apparatus. For example, the time value may correspond to the time between successive operating cycles of a pump assembly of the wound therapy apparatus. In such embodiments, the pump assembly of the apparatus may be configured to activate upon the pressure level inside the wound dressing reaching a first threshold level (e.g. an activation threshold level) and deactivate upon the pressure level inside the wound dressing reaching a second threshold level (e.g. a deactivation level).

The input signal received by the control system may be indicative of a time value corresponding to the time between the end of a first operating cycle of the pump assembly (e.g. upon deactivation of the pump assembly) and the start of a second operating cycle of the pump assembly (e.g. upon subsequent activation of the pump assembly). In such embodiments, the parameter comprises the time taken for the pressure level within the wound dressing to change (e.g. drop) from the second threshold level to the first threshold level when the pump assembly is deactivated.

In some embodiments, the control system may be configured to compare the time value, calculated by measuring the time between the end of a first operating cycle of the pump assembly and the start of a second operating cycle of the pump assembly, with a second time value corresponding to the length of time of an operating cycle (e.g. the second operating cycle) of the pump assembly. In such embodiments, the control system may be operable to determine a parameter in the form of a ratio of the time for which the pump assembly is not operating and the time for which the pump assembly is operating. The ratio may be determined as a percentage of time for which the pump assembly is not operating during a single duty cycle of the pump assembly. The control system may be configured to determine the ratio as a percentage of time for which the pump assembly is not operating during a single duty cycle of the pump assembly.

According to a further aspect of the invention there is provided a control system for monitoring operation of a pressure gradient wound therapy apparatus, the control system comprising one or more controllers, the control system being configured to: receive an input signal indicative of a first pressure value, the first pressure value corresponding to the pressure within an applied wound dressing within a first time period; compare the first pressure value with a second pressure value corresponding to the pressure within the applied wound dressing within a preceding time period to determine a rate of change of pressure within the applied wound dressing, the rate of change of pressure corresponding to a leak level; compare the determined rate of change of pressure to a plurality of leak thresholds to categorise the leak level into one of a plurality of leak level categories; and output a control signal to control the wound therapy apparatus in accordance with one or more predetermined actions in dependence on the leak level category.

The following optional statements apply to any aspect described herein.

In embodiments, the one or more controllers the one or more controllers collectively comprise: at least one electronic processor having an electrical input for receiving the input signal. The one or more controllers may collectively comprise at least one electronic memory device electrically coupled to the at least one electronic processor and having instructions stored therein. The at least one electronic processor may be configured to access the at least one memory device and execute the instructions thereon so as to generate the control signal for controlling the wound therapy apparatus in dependence on the leak level category.

In embodiments the control system may be configured to categorise the leak level into leak level categories corresponding to one or more of: a no leak category; a minor leak category; and a major leak category. One or more further leak level categories may be used, such as a medium or intermediate leak level category between a minor leak category and a major leak category.

The or each pressure value (e.g. the first and/or second pressure values) may correspond to absolute pressure values within the applied wound dressing. In embodiments, the first and second pressure values may correspond to relative pressure values within the applied wound dressing, which may be relative to atmospheric pressure or to a desired/optimum pressure value for the wound dressing.

The control system may be configured to output a control signal to control an operating level of a component of the wound therapy apparatus in dependence on the leak level category. The component may comprise a pump assembly of the wound therapy apparatus. In such embodiments, the operating level may comprise a power output or motor speed of the pump assembly, for example. In some embodiments the control system may be configured to output a control signal to activate the pump assembly in response to an increase in the leak level—e.g. a change in a leak level such that it is categorised in a leak level category corresponding to a higher leak level. The control system may be configured to output the control signal to increase the power output/motor speed of the pump assembly in response to an increase in the leak level. Advantageously, the control system may be configured to control the pump assembly to overcome the identified leak and thereby reach a desired pressure level within the wound dressing. In further embodiments, the control system may be configured to output a control signal to decrease or deactivate operation of the pump assembly in response to an increase in the leak level. For instance, in some cases the leak level may be such that the pump assembly cannot act to reach the desired pressure level within the wound dressing, or in doing so would result in excessive energy consumption or wear on components of the pump assembly. Accordingly, it may be advantageous to cease operation of the pump assembly to prevent excess energy consumption and/or damage to the pump assembly itself.

The control system may be configured to control output of an indication to a user of the apparatus in dependence on the leak level category. The indication may include one or more of illuminating a light, controlling an associated display, and/or activating an alert to inform the user of the apparatus of the presence of a leak. The indication may comprise information indicative of the leak level category. For example, the indication may inform the user of the leak level category directly. The control system may be configured to control output of instructions for a user to address the leak in dependence on the leak level category—e.g. increase pump output, deactivate pump, re-seal/replace wound dressing. The user may then act in response to such an output. In some embodiments the indication may be purely informative and be indicative of one or more actions taken in dependence on the leak level category, for instance in embodiments wherein the control system is configured to automatically control operation of the pump assembly in dependence on the leak level category.

In embodiments, the control system is configured to control the wound therapy apparatus in accordance with one or more different instructions in dependence on the leak level category. For example, in some embodiments the control system is configured output a control signal to: control the wound therapy apparatus in accordance with a first set of instructions in dependence on a determination of a leak level in a first leak level category; and control the wound therapy apparatus in accordance with a second set of instructions in dependence on a determination of a leak level in a second leak level category. The control system may further be configured to control the wound therapy apparatus in accordance with a third and/or fourth set of instructions in dependence on a determination of a leak level in a third or fourth leak level category. For instance, in embodiments the control system is configured to: take no action in dependence on a determination of a leak level in a no leak category; output a first control signal for controlling output of an indication to a user of the apparatus in dependence on a determination of a leak level in a minor leak level category; output a second control signal for controlling an operating level of a pump assembly of the wound therapy apparatus in dependence on a determination of a leak level in a medium leak level category; and output a third control signal to prevent operation of the pump assembly in dependence on a determination of a leak level in a major leak level category.

The control system may be configured to control the wound therapy apparatus in accordance with a combination of different predetermined actions in dependence on the leak level category. For example, for some leak level categories, the control system may be configured to control an operating level of a pump assembly of the wound therapy apparatus and output an indication to the user indicative of the leak level and/or the change in operating level of the pump assembly. For instance, in embodiments the control system is configured to: take no action in dependence on a determination of a leak level in a no leak category; control an operating level of a pump assembly of the wound apparatus and output a first indication to a user of the apparatus in dependence on a determination of a leak level in a minor leak level category; control an operating level of a pump assembly of the wound therapy apparatus and output a second indication to a user of the apparatus in dependence on a determination of a leak level in a medium leak level category; and prevent operation of the pump assembly and output a third indication to a user of the apparatus in dependence on a determination of a leak level in a major leak level category. The first, second and/or third indications may be different. For instance, for a low level the indication may comprise a visual indication, only. However, for higher leak level categories the indication may comprise both a visual and audible indication such as an alert or alarm.

The control system may be configured to obtain a plurality of pressure values within the first time period. In such embodiments, the control system may be configured to obtain an average of the plurality of pressure values obtained within the first time period to obtain the first pressure value. Similarly, the control system may be configured to obtain a plurality of pressure values within the preceding time period. In such embodiments, the control system may be configured to obtain an average of the plurality of pressure values obtained within the preceding time period to obtain the second pressure value.

The first time period may comprise a time period of at least 10 ms, or at least 20 ms, or at least 50 ms, or at least 100 ms, or at least 150 ms, for example. The first time period may comprise a time period of no more than 500 ms, or no more than 250 ms, or no more than 200 ms, or no more than 150 ms, or no more than 100 ms, or no more than 50 ms, for example. In some embodiments the first time period may comprise a time period of approximately 200 ms.

The control system may be configured to obtain a plurality of pressure values at intervals within the first time period. For example, the control system may be configured to obtain pressure values at intervals of approximately 10 ms. The control system may be configured to obtain at least 1, or at least 2, or at least 5, or at least 10, or at least 15, or at least 20, or at least 50 pressure values within the first time period.

The preceding time period may comprise a time period of at least 10 ms, or at least 20 ms, or at least 50 ms, or at least 100 ms, or at least 150 ms, for example. The preceding time period may comprise a time period of no more than 500 ms, or no more than 250 ms, or no more than 200 ms, or no more than 150 ms, or no more than 100 ms, or no more than 50 ms, for example. In some embodiments the preceding time period may comprise a time period of approximately 200 ms. In embodiments, the preceding time period is equal in length to the first time period.

The control system may be configured to obtain a plurality of pressure values at intervals within the preceding time period. For example, the control system may be configured to obtain pressure values at intervals of approximately 10 ms. The control system may be configured to obtain at least 1, or at least 2, or at least 5, or at least 10, or at least 15, or at least 20, or at least 50 pressure values within the preceding time period.

The control system may be configured to compare the rate of change of pressure to a first leak threshold and categorise the corresponding leak level in a first leak level category if the rate of change of pressure is below the first leak threshold. The control system may be configured to compare the rate of change of pressure to a second leak threshold and categorise the corresponding leak level in a second leak level category if the rate of change of pressure is below the second leak threshold but above the first leak threshold. The invention may extend to a control system which is configured to compare the rate of change of pressure to a third leak threshold and categorise the corresponding leak level in a third leak level category if the rate of change of pressure is below the third leak threshold but above the second leak threshold. The control system may be configured to categorise the corresponding leak level in a fourth leak level category if the rate of change of pressure is above the third leak threshold. The first leak level category may be a no leak category. The second leak level category may be a minor leak level category. The third leak level category may be a medium leak level category. The fourth leak level category may be a major leak level category.

In embodiments, the control system is configured to compare the determined rate of change of pressure (i.e. the rate of change of pressure corresponding to the first pressure value) with a rate of change of pressure observed at one or more preceding time periods. For example, the control system may be configured to obtain an average rate of change of pressure observed at a plurality of preceding time periods, and compare the determined rate of change of pressure with the average rate of change of pressure observed at the plurality of preceding time periods. The control system may be configured to identify the presence of a leak and/or determining a leak level in dependence on this comparison.

The control system may be configured to determine a rate of change of pressure within the wound dressing as follows:

$$Rate of change of pressure = \vert \frac{{P}_{m} - {P}_{m - 1}}{{T}_{m} - {T}_{m - 1}} \vert \qquad [ Equation 1 ]$$

where Pₘ is the first pressure value, Pₘ₋₁ is the second pressure value, Tₘ corresponds to a time stamp of the first time period, and Tₘ₋₁ corresponds to a time stamp of the preceding time period.

The control system may be configured to monitor operation of the wound therapy apparatus only when a pump assembly of the wound therapy apparatus is inactive—i.e. when it is not providing a source of positive or negative pressure to the wound dressing. In such embodiments, the control system may be configured to receive an input signal from the wound therapy apparatus indicative of the operational state of the pump assembly.

The control system may be configured to obtain the or each pressure value (e.g. the first and/or second pressure values) from one or more sensors (e.g. pressure sensors) associated with the wound dressing. The one or more sensors may be embedded within the wound dressing, and/or may be arranged about a periphery of the wound dressing. In embodiments, the one or more sensors may comprise micro-electromechanical system (MEMS) or nano-electromechanical system (NEMS) sensors. The one or more sensors may be arranged as a strip of sensors about a periphery of a wound dressing. The one or more sensors may be associated with a pump assembly of the wound therapy apparatus. The one or more sensors may form part of the control system.

In embodiments, the control system is for monitoring operation of a negative pressure wound therapy apparatus. In other embodiments, the control system is for monitoring operation of a positive pressure wound therapy apparatus.

According to another aspect of the invention there is provided a wound therapy apparatus arranged to perform a method as described herein and/or comprising the control system as described herein.

In embodiments, the wound therapy apparatus comprises a negative pressure wound therapy apparatus. In other embodiments, the wound therapy apparatus comprises a positive pressure wound therapy apparatus.

The wound therapy apparatus may comprise a wound dressing. The wound dressing may include a dressing body formed of an absorbent material which may be positioned in contact with a wound, in use. The dressing body may be configured to absorb exudate from the wound, aided by the action of the pump assembly. The dressing body may be formed of a hydrocolloid material which may gel in the presence of an exudate. The hydrocolloid material may comprise a layer or multiple layers of gelling fibres and absorbent materials. The outer surface of the dressing may be constructed of a thin film layer (e.g. a polyurethane) enabling moisture vapour to exit the dressing at an increased rate. This combination would allow the wound therapy apparatus to manage fluid without the need of a canister. This may be referred to as a “canister-less” or “canister free” system. In a variant, the wound dressing may be fluidly connected to a canister into which exudate removed from the wound may be withdrawn. The dressing may include an adhesive layer for providing a seal between the dressing and the user's skin, in use. The adhesive layer may define an interior region of the wound dressing.

The wound dressing may have a thickness between 1 mm to 20 mm, or 2 mm to 10 mm, or 3 mm to 7 mm, for example. The wound dressing may be comprised of one or more layers including an outer cover layer, an absorbent layer, a gel-forming fibre, an adhesive layer, a wound contact layer, a distribution layer, and combinations thereof. The wound dressing may include one or more absorbent layer(s). The absorbent layer may be a foam or a superabsorbent. If foam is used, the foam may also act as a distribution layer. The wound dressing may comprise an outer cover layer and one or more absorbent layer(s) and a silicone gel wound contact layer. The wound dressing may comprise an outer cover layer and one or more absorbent layer(s) in combination with a gel-forming fibre. The gel-forming fibre typically is in direct contact with the wound, and thus no additional wound contact layer is required i.e., a silicone gel wound contact layer does not require a silicone gel layer.

Gel-forming fibres include hygroscopic fibres which upon the uptake of wound exudate become moist slippery or gelatinous. The gel forming fibres can be of the type which retain their structural integrity on absorption of exudate or can be of the type which lose their fibrous form and become an amorphous or structureless gel. The gel forming fibres are preferably sodium carboxymethylcellulose fibres, chemically modified cellulosic fibres, alkyl sulphonate modified cellulosic fibres such as those described in WO2012/061225, pectin fibres, alginate fibres, chitosan fibres, hyaluronic acid fibres, or other polysaccharide fibres or fibres derived from gums. The cellulosic fibres preferably have a degree of substitution of at least 0.05 carboxymethyl groups per glucose unit. The gel forming fibres preferably have an absorbency of at least 2 grams 0.9% saline solution per gram of fibre (as measured by the free swell method).

The gel forming fibres are preferably chemically modified cellulosic fibres in the form of a fabric and in particular carboxymethylated cellulose fibres as described in PCT WO00/01425 to Azko Nobel UK Ltd, and can be provided by a layer of gel forming fibres preferably located in a port of the cover layer or as a layer of fibres in a conduit of the wound dressing. When present in the conduit, the layer of fibres can also serve to keep the conduit open to the passage of fluid in the event that the conduit is kinked or otherwise restricted by being lain on or leaned on by the user. The carboxymethylated cellulosic fabrics preferably have a degree of substitution between 0.12 to 0.35 as measured by IR spectroscopy (as defined in WO00/01425) more preferably a degree of substitution of between 0.20 and 0.30 and are made by carboxymethylating a woven or non-woven cellulosic fabric such that the absorbency is increased. Particular preferred fabrics have an absorbency of between 10 g/g of sodium/calcium chloride as defined above to 30 g/g of sodium/calcium chloride as measured by the method described in BS EN 13726-1 (2002) “Test methods for primary wound dressings”, section 3.2 “Free swell absorptive capacity”. Particularly preferred fabrics have an absorbency of 15 g/g to 25 g/g and most preferred of 15 g/g to 20 g/g of sodium/calcium chloride as measured by the method defined above.

The cellulosic fabric preferably consists solely of cellulosic fibre but may contain a proportion of non-cellulosic textile fibre or gel forming fibre. The cellulosic fibre is of known kind and may comprise continuous filament yarn and/or staple fibre. The carboxymethylation is generally performed by contacting the fabric with an alkali and a carboxymethylating agent such a chloracetic acid in an aqueous system. The fabric is preferably of a non-woven type to reduce shedding in the wound on cutting the dressing. Preferably the fabric is hydroentangled and thus comprises a series of apertures on a microscopic scale.

Where present, the absorbent layer of the wound dressing is capable of absorbing exudate from the wound and allowing the passage of fluid through it. The absorbent layer can comprise any absorbent capable of absorbing exudate while allowing the passage of fluid through it, such as a foam, sponge or fibre-based material, preferably the absorbent layer is provided by gel forming fibres of the same type or of a different type as those discussed above. The gel-forming fibres are hygroscopic fibres which upon the uptake of wound exudate become moist slippery or gelatinous and thus reduce the tendency for the surrounding fibres to adhere to the wound. The gel forming fibres are preferably spun sodium carboxymethylcellulose fibres, chemically modified cellulosic fibres, alkyl sulphonate modified cellulosic fibres such as those described in WO2012/061225, pectin fibres, alginate fibres, chitosan fibres, hyaluronic acid fibres, or other polysaccharide fibres or fibres derived from gums. The cellulosic fibres preferably have a degree of substitution of at least 0.05 carboxymethyl groups per glucose unit and more preferably are lightly substituted so that the absorbency of the fibres is limited. The gel forming fibres preferably have an absorbency of at least 2 grams 0.9% saline solution per gram of fibre (as measured by the method described above) but less than 30 grams 0.9% saline solution per gram of fibre. The gel forming fibres are preferably carboxymethylated cellulose fibres as described in PCT WO00/01425 to Azko Nobel UK Ltd which describes lightly carboxymethylated cellulose fabrics. The gel forming fibres are preferably lightly carboxymethylated in order to reduce the tendency of the absorbent layer to gel block and block the pathway for fluid from the wound, e.g. through the absorbent layer, the port and to a distal end of the conduit.

Preferably the absorbent layer, where present, is provided with fenestrations to aid the application of negative pressure to the wound and maintain the pathway for fluid from the wound, through the absorbent layer. Typically, however, fenestrations are only provided in internal absorbent layers. External absorbent layers, including those in direct contact with the wound, generally do not have mechanically added fenestrations, however, they may include openings between the fibres.

Although the absorbent layer can be in direct contact with the wound, preferably the dressing comprises a wound contact layer, positioned between the wound and the absorbent layer. The wound contact layer may be capable of absorbing exudate from the wound and transmitting it to the absorbent layer. Like the absorbent layer, the wound contact layer may be capable of allowing the passage of fluid through it so that pressure (either positive or negative) may applied to the wound and the pathway for fluid/exudate from the wound to the distal end of the conduit may be maintained.

The wound contact layer may include gel-forming fibres (e.g. of the type discussed herein), or a silicone gel, for example.

Preferably the wound contact layer comprises gel-forming fibres. The gel-forming fibres may be the same or a similar type to those comprising the absorbent layer but the wound contact layer may be strengthened to increase its integrity and that of the dressing. For example, the wound contact layer may be of the type described in EP 1904011 and comprise gel-forming fibres in the form of a mat with lines of longitudinal stitching made of cellulose or nylon or polyolefin yarn to increase the integrity of the layer. Preferably the wound contact layer is porous to maintain the pathway for fluid/exudate from the wound to the distal end of the conduit.

An outer cover layer of the dressing is provided as a bacterial and viral barrier layer which preferably resists the ingress of liquid and air but allows moisture vapour transmission. In this way the outer cover layer enhances the overall fluid handling capacity of the dressing by allowing for the escape of moisture vapour through the cover while enabling the application of pressure (either positive or negative) to the wound. The outer cover layer is for instance a layer having a MVTR of at least 10,000 g m⁻² per 24 hours or in the range of from 10,000 gm⁻² to 50,000 g m⁻² per 24 hours measured by the method described in BS EN 13726-2 2002 “Test methods for primary wound dressings Part 2 Moisture vapour transmission rate of permeable film dressings”. The cover layer may be in the form of a film of polyurethane, for example Epurex 912 T/129 manufactured by Covestro or Inspire 2350 manufactured by Coveris or Medifilm 426 manufactured by Mylan.

The cover layer can be provided with a port for connection to the conduit. The port is preferably located in the cover layer and overlies the absorbent layer towards the periphery of the absorbent layer so that it is not directly in vertical alignment with the centre of the dressing (or the wound when in use). This assists in the spread of exudate across the full extent of the absorbent layer.

The conduit of the dressing is preferably a transparent passageway secured to the outside of the cover layer at the proximal end of the conduit so as to surround the port in the cover layer from above. The conduit of the dressing may comprise a connector, at its distal end, for connecting the dressing to a source of pressure (either positive or negative), for example a pump. Preferably the connector is a luer lock to facilitate secure connection to the pump and to maintain the pressure within the wound dressing while the pump is temporarily disconnected. The connector preferably comprises a one-way lock to assist in the maintenance of the applied pressure. To resist collapse, the conduit may comprise an internal cylinder of nylon fibres to maintain openness of the conduit to fluid.

The dressing may further comprise a distribution layer, e.g., a pressure distribution layer, located between the absorbent layer and the outer cover layer which is gas and liquid permeable and particularly moisture vapour permeable and serves to aid access of exudate to a greater area of the absorbent layer by allowing it to spread under the distribution layer. The distribution layer also serves to even out the negative pressure applied to the wound over the whole dressing. The distribution layer preferably distributes exudate and negative pressure over the dressing. In this way, uptake of exudate by the absorbent layer is maximised before the exudate leaves the absorbent layer and activates the indicator means and the transfer of negative pressure to the wound is optimised. The distribution layer is preferably a foam layer such as a polyester foam of the type XD4200AS manufactured by Caligen or another suitable reticulated foam.

The dressing may also comprise additional optional layers such as an adhesive layer for adhering the dressing to the skin surrounding the wound to form a fluid tight seal. The adhesive layer may be applied to the side of dressing closest to the wound and may be provided with perforations to assist transport of exudate and fluid through the dressing. The adhesive layer may also be applied to any of the other layers to provide an island configuration such as to the cover layer.

The pump assembly may be fluidly connected to an interior region of the wound dressing, for introducing and/or removing gas from within the wound dressing to control the pressure therein.

The wound therapy apparatus may comprise one or more sensors. The one or more sensors may be embedded within or otherwise associated with one or more components of a wound dressing of the wound therapy apparatus. In embodiments, the one or more sensors may comprise micro-electromechanical system (MEMS) or nano-electromechanical system (NEMS) sensors. The one or more sensors may be arranged as a strip of sensors about a periphery of a wound dressing. The one or more sensors may comprise a pressure sensor. The one or more sensors may be associated with another part of the wound therapy apparatus. For example, the one or more sensors may be located in a housing with the pump, and/or arranged between a pump and a wound dressing of the wound therapy apparatus, e.g. associated with a conduit or tube connecting the wound dressing and the pump, or associated with a canister forming part of the wound therapy apparatus.

According to an aspect of the invention there is provided computer software which, when executed by one or more processors, causes performance of a method in accordance with a preceding aspect of the invention.

According to an aspect of the invention there is provided a computer readable medium comprising the computer software of a preceding aspect of the invention. Optionally, the computer readable medium comprises a non-transitory computer readable medium.

### DESCRIPTION OF THE FIGURES

In order that the invention may be more clearly understood one or more embodiments thereof will now be described, by way of example only, with reference to the accompanying drawings, of which:

FIG. 1A is a flowchart illustrating an embodiment of a method of the invention;

FIG. 1B is a flowchart illustrating a further embodiment of a method of the invention;

FIG. 2 is a further flowchart illustrating an embodiment of a method of the invention;

FIG. 3 is a schematic view illustrating an embodiment of a control system of the invention;

FIG. 4 is a schematic view illustrating an embodiment of a wound therapy apparatus of the invention;

FIG. 5 is a flowchart illustrating a further embodiment of a method of the invention; and

FIG. 6 is a flowchart further illustrating the method shown in FIG. 5.

### DETAILED DESCRIPTION

Embodiments disclosed herein relate to apparatus and methods of treating a wound with reduced or positive pressure (typically negative pressure), including pump and wound dressing components and devices. The devices and components may include a wound overlay and packing materials, which may be collectively referred to interchangeably herein as “dressings” or “wound dressings”.

As disclosed herein the present invention may comprise an apparatus for providing pressure gradient wound therapy to a wound, comprising: the technology disclosed herein, a wound dressing described herein; and a source of positive or negative pressure.

As used herein the expression “wound” may include an injury to living tissue may be caused by a cut, blow, or other impact, typically one in which the skin is cut or broken. A wound may be a chronic or acute injury. Acute wounds occur as a result of surgery or trauma. They move through the stages of healing within a predicted timeframe. Chronic wounds typically begin as acute wounds. The acute wound can become a chronic wound when it does not follow the healing stages resulting in a lengthened recovery. It is believed that the transition from acute to chronic wound can be due to a patient being immuno compromised.

Chronic wounds may include for example: venous ulcers (such as those that occur in the legs), which account for the majority of chronic wounds and mostly affect the elderly, diabetic ulcers (for example, foot or ankle ulcers), peripheral arterial disease, pressure ulcers, or epidermolysis bullosa (EB).

Examples of other wounds include, but are not limited to, abdominal wounds or other large or incisional wounds (either as a result of surgery, trauma, stemiotomies, fasciotomies, or other conditions), dehisced wounds, acute wounds, chronic wounds, subacute and dehisced wounds, traumatic wounds (such as from orthopaedic trauma), flaps and skin grafts, lacerations, abrasions, contusions, burns, diabetic ulcers, pressure ulcers, stoma, surgical wounds, trauma and venous ulcers, broken bones or the like.

Wounds may also include a deep tissue injury. Deep tissue injury is a term proposed by the National Pressure Ulcer Advisory Panel (NPUAP) to describe a unique form of pressure ulcers. These ulcers have been described by clinicians for many years with terms such as purple pressure ulcers, ulcers that are likely to deteriorate and bruises on bony prominences.

The technology disclosed can be used on an acute or chronic wound.

Wounds are believed to be more susceptible to infection under the following circumstances. If the wounds are chronic wounds, or if an object which caused the wound was dirty or contained bacteria, or from a bite, or contains remnant or a whole object that caused the wound, or a wound that is large or deep, or jagged edges to the wound, or elderly, or chronic because by their nature a wound site is open; and/or if the patient has: diabetes type 1 or type 2, is elderly, or has a compromised immune system.

Pressure gradient wound therapy may also be useful for treating second- and third-degree burns, as well as being useful for laparotomy surgery i.e., a large incision through an abdominal wall to gain access into the abdominal cavity.

In general, the invention relates to a method 10, 10′ and a control system 100 for monitoring (and in embodiments controlling) operation of a pressure gradient wound therapy apparatus, e.g. wound therapy apparatus 200.

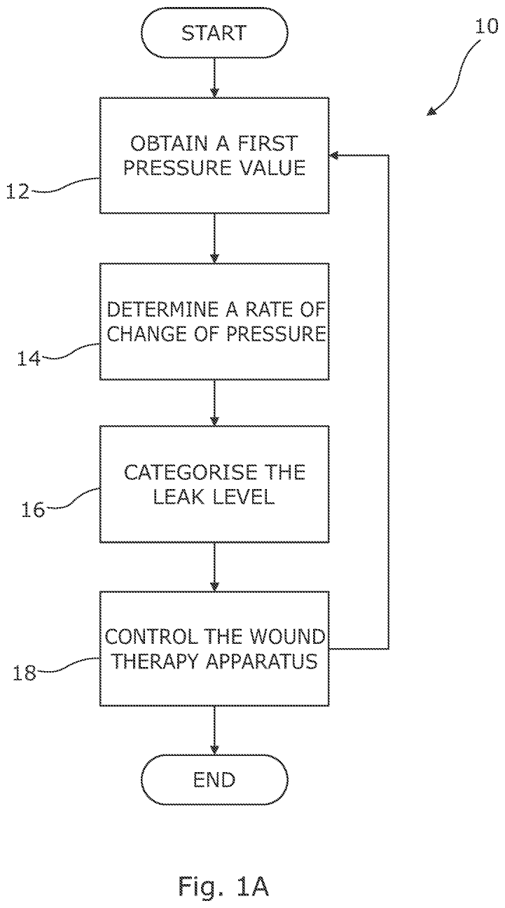

FIG. 1A illustrates a first embodiment of a method 10.

At step 12, a first pressure value corresponding to the pressure within an applied wound dressing within a first time period is obtained. The first pressure value is obtained using one or more sensors, for example pressure sensors associated with a wound dressing of the wound therapy apparatus. The one or more sensors may be embedded within the wound dressing, and/or may be arranged about a periphery of the wound dressing, and/or may be associated with a pump assembly of the wound therapy apparatus.

The first pressure value can be an absolute pressure value within the applied wound dressing, or a relative pressure value within the applied wound dressing for example with reference to atmospheric pressure or to a desired or optimum pressure value for the wound dressing.

In an extension of method 10, step 12 comprises obtaining a plurality of pressure values within the first time period. Here, an average of the plurality of pressure values is calculated to obtain the first pressure value.

At step 14, the method 10 comprises comparing the first pressure value with a second pressure value corresponding to the pressure within the applied wound dressing within a preceding time period.

Again, the second pressure value is obtained using the one or more sensors. Similarly, the second pressure value can be an absolute pressure value within the applied wound dressing, or a relative pressure value within the applied wound dressing for example with reference to atmospheric pressure or to a desired or optimum pressure value for the wound dressing.

By performing this comparison at step 14, the method 10 is used to determine a rate of change of pressure within the applied wound dressing, specifically using Equation 1, repeated here as follows:

$$Rate of change of pressure = \vert \frac{{P}_{m} - {P}_{m - 1}}{{T}_{m} - {T}_{m - 1}} \vert \qquad [ Equation 1 ]$$

where Pₘ is the first pressure value, Pₘ₋₁ is the second pressure value, Tₘ corresponds to a time stamp of the first time period, and Tₘ₋₁ corresponds to a time stamp of the preceding time period.

The rate of change of pressure corresponds to a leak level of the wound dressing. For instance, a high rate of change of pressure may correspond to the presence of a significant leak.

At step 16, the method 10 comprises comparing the determined rate of change of pressure to a plurality of leak thresholds. In doing so, the method may be used to categorise the leak level into one of a plurality of leak level categories. For instance, the plurality of leak level categories can include a no leak category; a minor leak category; and a major leak category. In the illustrated embodiment, the method 10 comprises at step 16 categorising the leak level into one of four categories, corresponding to a no leak category, a minor leak category, a medium leak category and a major leak category.

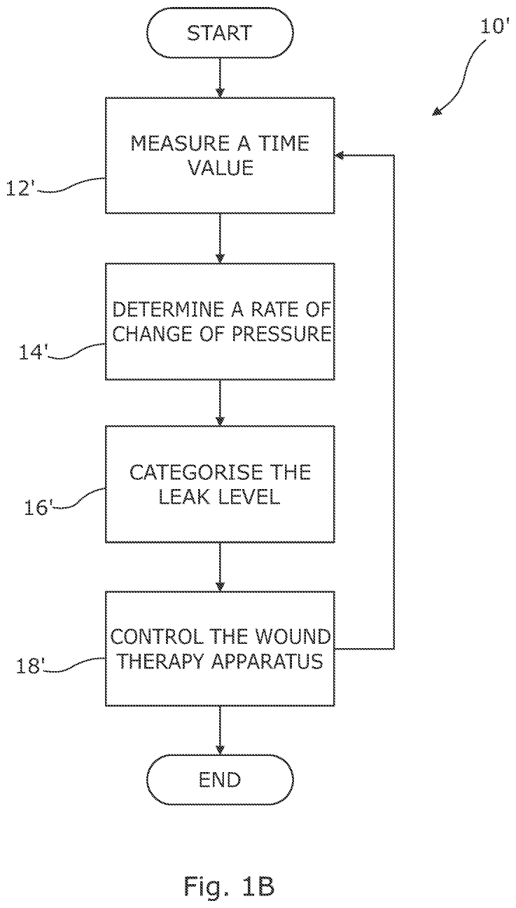

FIG. 2 illustrates the categorisation step 16 of method 10 in more detail.

Specifically, at step 20a the determined rate of change of pressure is compared to a first leak threshold. If the comparison indicates that the determined rate of change of pressure is less than or equal to the first leak threshold, the method 10 moves to step 20b where the leak level is categorised as no leak. Method 10 then moves to step 20c wherein the appropriate action is identified as “no action”.

If, however, the comparison at step 20a indicates that the determined rate of change of pressure is greater than the first leak threshold, the method 10 moves to step 22a where the determined rate of change of pressure is compared to a second leak threshold. If the comparison indicates that the determined rate of change of pressure is less than or equal to the second leak threshold, the method 10 moves to step 22b where the leak level is categorised as a minor leak. Method 10 then moves to step 22c wherein the appropriate action is identified as to output an indication of the leak to a user as described herein.

If, however, the comparison at step 22a indicates that the determined rate of change of pressure is greater than the second leak threshold, the method 10 moves to step 24a where the determined rate of change of pressure is compared to a third leak threshold. If the comparison indicates that the determined rate of change of pressure is less than or equal to the third leak threshold, the method 10 moves to step 24b where the leak level is categorised as a medium leak. Method 10 then moves to step 24c wherein the appropriate action is identified as to control a pump assembly of the wound therapy apparatus as described herein, for example to compensate for the leak.

If, however, the comparison at step 24a indicates that the determined rate of change of pressure is greater than the third leak threshold, the method 10 moves to step 26a, where it is confirmed that determined rate of change of pressure is greater than the third leak threshold. If so, the method 10 moves to step 26b where the leak level is categorised as a major leak. In such instances, an appropriate action is identified which here comprises preventing further operation of the pump assembly. This prevents the pump assembly being operated where it would not be able to provide the required pressure level due to the significance of the leak, thereby preventing excessive energy consumption and/or wear.

Following the categorisation step 16, the method 10 moves to step 18 which comprises controlling the wound therapy apparatus in accordance with one or more predetermined actions in dependence on the leak level category—e.g. the appropriate actions identified in step 16.

FIG. 1B illustrates a second embodiment of a method 10′ in accordance with the invention.

At step 12′, a time value is measured. Specifically, the method 10′ comprises measuring a time value corresponding to the time between successive operating cycles of a pump assembly of the wound therapy apparatus. Here, the pump assembly of the apparatus is configured to activate upon the pressure level inside the wound dressing reaching a first threshold level (e.g. an activation threshold level) and deactivate upon the pressure level inside the wound dressing reaching a second threshold level (e.g. a deactivation level). Accordingly, at step 12′ a time value corresponding to the time between the end of a first operating cycle of the pump assembly (e.g. upon deactivation of the pump assembly) and the start of a second operating cycle of the pump assembly (e.g. upon subsequent activation of the pump assembly) is measured. This time value then corresponds to the time taken for the pressure level within the wound dressing to change (e.g. drop) from the second threshold level to the first threshold level when the pump assembly is deactivated.

At step 14′, the measured time value is compared with a second time value corresponding to the length of time of an operating cycle of the pump assembly to determine a rate of change of pressure in the applied wound dressing. Specifically, the measured time value is compared with the second time value to obtain a ratio of the time for which the pump assembly is not operating and the time for which the pump assembly is operating, the ratio being indicative of a rate of change of pressure in the applied wound dressing.

Step 16′ comprises comparing the determined rate of change of pressure to a plurality of leak thresholds. This is performed in the same way as step 16 of method 10 as discussed above and illustrated in FIG. 2.

Following the categorisation step 16′, the method 10′ moves to step 18′ which comprises controlling the wound therapy apparatus in accordance with one or more predetermined actions in dependence on the leak level category—e.g. the appropriate actions identified in step 16′.

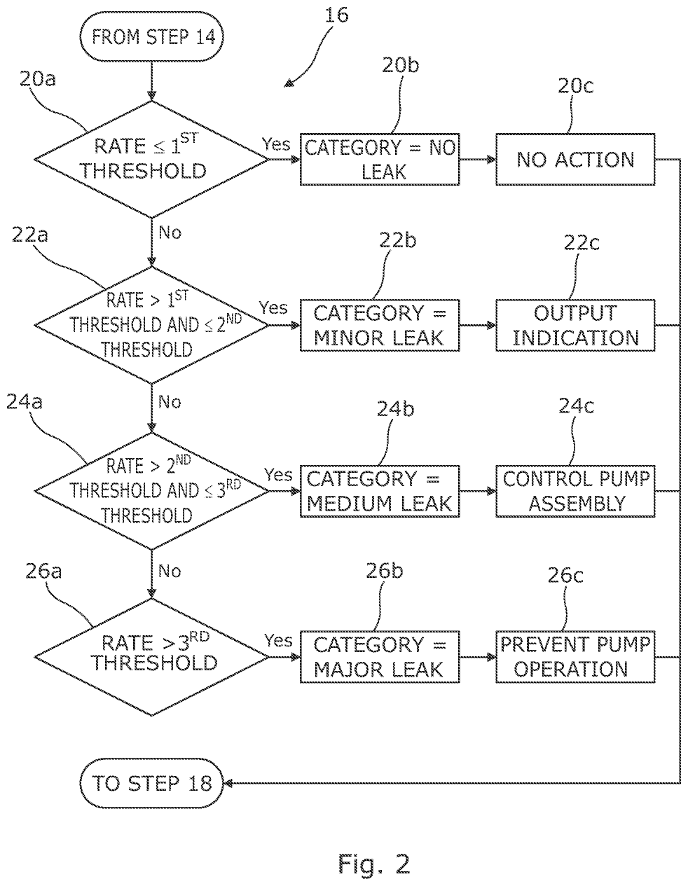

FIG. 3 illustrates an embodiment of a control system 100, which includes a controller 102 having a processor 104.

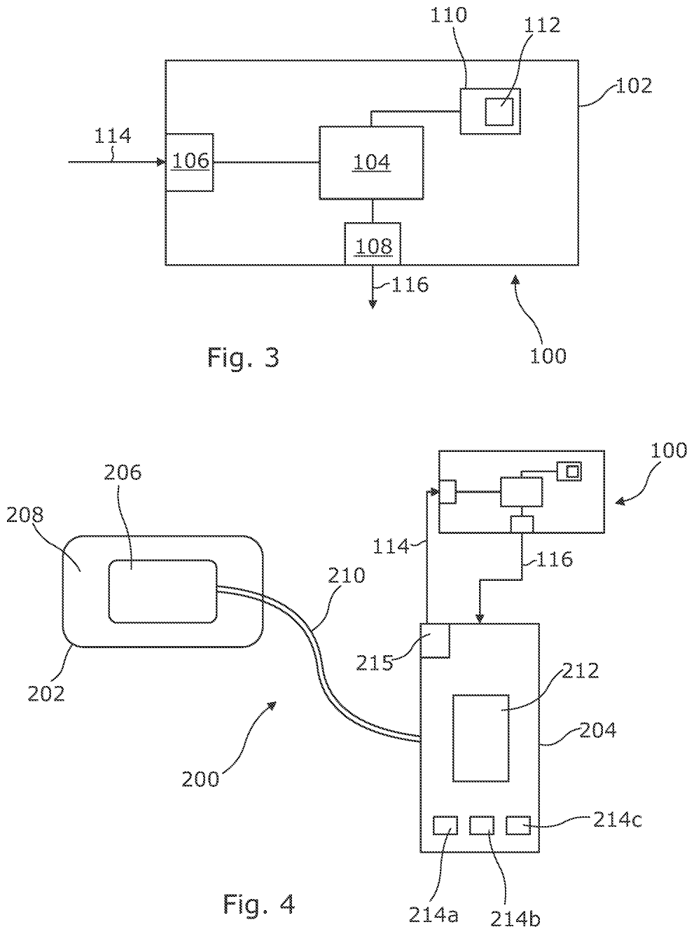

The processor 104 is operably coupled to an electrical input 106 for receiving an input signal 114. In use, the input signal 114 comprises data indicative of a first pressure value corresponding to the pressure within an applied wound dressing within a first time period. The input signal 114 may be received directly from a pressure sensor 215 (FIG. 4) associated with the wound dressing.

The controller includes memory device 110 electrically coupled to the processor 104 and includes instructions 112 stored therein. The instructions 112 may relate to operating instructions for controlling the operation of the wound therapy apparatus. In use, the processor 104 is configured to access the memory device 110 and execute the instructions 112 in order to generate a control signal 116 for controlling operation of the wound therapy apparatus. The control signal 116 is output via electrical output 108.

As discussed, the controller 102 is configured to receive the input signal 114 from a pressure sensor 215 (FIG. 4) associated with the wound dressing. The processor 104 is configured to analyse the input signal 114 to determine a rate of change of pressure within the wound dressing. Specifically, the processor 104 is configured to extract a first pressure value from the input signal 114 and compare the first pressure value with a second pressure value corresponding to the pressure within the applied wound dressing within a preceding time period to determine a rate of change of pressure within the applied wound dressing. The rate of change of pressure is indicative of a leak level from the wound dressing, so can be used to determine the significance or severity of any leaks present.

The comparison may be performed as per equation 1—repeated here as follows:

$$Rate of change of pressure = \vert \frac{{P}_{m} - {P}_{m - 1}}{{T}_{m} - {T}_{m - 1}} \vert \qquad [ Equation 1 ]$$

where Pₘ is the first pressure value, Pₘ₋₁ is the second pressure value, Tₘ corresponds to a time stamp of the first time period, and Tₘ₋₁ corresponds to a time stamp of the preceding time period.

With the determined rate of change of pressure, the processor 104 is configure to compare the determined rate of change of pressure to a plurality of leak thresholds. The plurality of leak thresholds may be stored within the memory device 110, for example. On the basis of this comparison, the processor 104 categorises the leak level associated with the determined rate of change of pressure into one of a plurality of leak level categories. In the illustrated embodiment, the plurality of leak level categories include a no leak category; a minor leak category; a medium leak category; and a major leak category. As discussed herein, different actions may be appropriate depending on the leak level category.

Finally, the processor 104 is configured to generate and output a control signal 116 for controlling operation of the wound therapy apparatus in accordance with one or more predetermined actions. For example, depending on leak level category the processor 104 can output a control signal 116 for controlling operation of a pump assembly of the apparatus—e.g. to either control an operating characteristic of the pump, to prevent further operation of the pump, to output an indication to the user of the leak level or of the operating state of the pump, etc. —as appropriate for the level of leak from the wound dressing.

FIG. 4 illustrates a pressure gradient wound therapy apparatus 200 in accordance with the invention. The apparatus 200 includes a wound dressing 202 which may, for example be of the type available from ConvaTec Ltd. under the Avelle trade mark. The wound therapy apparatus 200 additionally includes a pump assembly 204 which may likewise, without pressure sensor 215, be of the type available from ConvaTec Ltd. under the Avelle trade mark.

The wound dressing 202 comprises a dressing body 206 and a peripheral adhesive layer 208. The dressing body 206 comprises an absorbent material and is positioned in contact with a wound, in use. The dressing body 206 is configured to absorb exudate from the wound, aided by the action of the pump assembly 204 creating a pressure differential between the interior of the wound dressing 202 and the surrounding environment. Here, the exudate is retained within the dressing body 206. Specifically, the dressing body 206 is formed of a hydrocolloid material which gels in the presence of exudate. This may be referred to as a “canister-less” system. In a variant, exudate removed from the wound may instead be withdrawn into an accompanying canister rather than being retained within the dressing body 206 itself. The adhesive layer 208 provides a seal between the dressing 202 and the user's skin, in use, defining an interior region of the wound dressing 202 about the wound.

The wound dressing 202 is fluidly connected to a pump 212 of the pump assembly 204 via a tube 210 which may likewise be of the type available from ConvaTec Ltd. under the Avelle trade mark. For positive pressure wound therapy, the pump 212 is configured to provide a source of air or other gas to be supplied to the interior portion of the wound dressing 202 to thereby increase the pressure within the wound dressing 202 relative to the surrounding environment. For negative pressure wound therapy, the pump 212 is configured to withdraw air from the interior portion of the wound dressing 202 to reduce the pressure within the wound dressing 202 relative to the surrounding environment.

The pump assembly 204 additionally includes indicators 214a, 214b, 214c consisting of lights which may be illuminated in dependence on the operational state of the pump 212 or indeed under instruction from the control system 100.

The apparatus 200 is controllable via control system 100.

Specifically, electrical input 106 of the controller 102 is operatively coupled to a pressure sensor 215 associated with the wound dressing 202. The pressure sensor 215 is provided within the pump assembly 204 and is configured to determine the pressure level within the wound dressing 202. The input signal 114 received from the pressure sensor 215 comprises data indicative of a first pressure value corresponding to the pressure within the wound dressing 202.

As discussed above, the processor 104 is configured to analyse the input signal 114 to determine a rate of change of pressure within the wound dressing 202 and compare the first pressure value with a second pressure value corresponding to the pressure within the applied wound dressing 202 within a preceding time period to determine a rate of change of pressure within the applied wound dressing 202 corresponding to a leak level from the wound dressing 202. From this, the leak level is then categorised and depending on the determined category for the observed leak level, the processor 104 is configured to generate and output the control signal 116 to the pump assembly 204 to control operation of the pump assembly 204 in accordance with one or more predetermined actions. For instance, the processor 104 can output a control signal 116 to an operating characteristic of the pump 212—e.g. to moderate its power output or a speed of a motor associated with the pump 212—or to prevent further operation of the pump 212. Additionally or alternatively, the processor 104 can output the control signal 116 to the pump assembly 204 to control operation of the indicators 214a, 214b, 214c to indicate to the user of the leak level or of the operating state of the pump 212.

Although shown separate in FIG. 4, it will be appreciated that in some embodiments two or more of the control system 100, wound dressing 202 and pump assembly 204 may be provided as a single unit, e.g. with the control system 100 and/or pump assembly 204 integrated within the wound dressing 202.

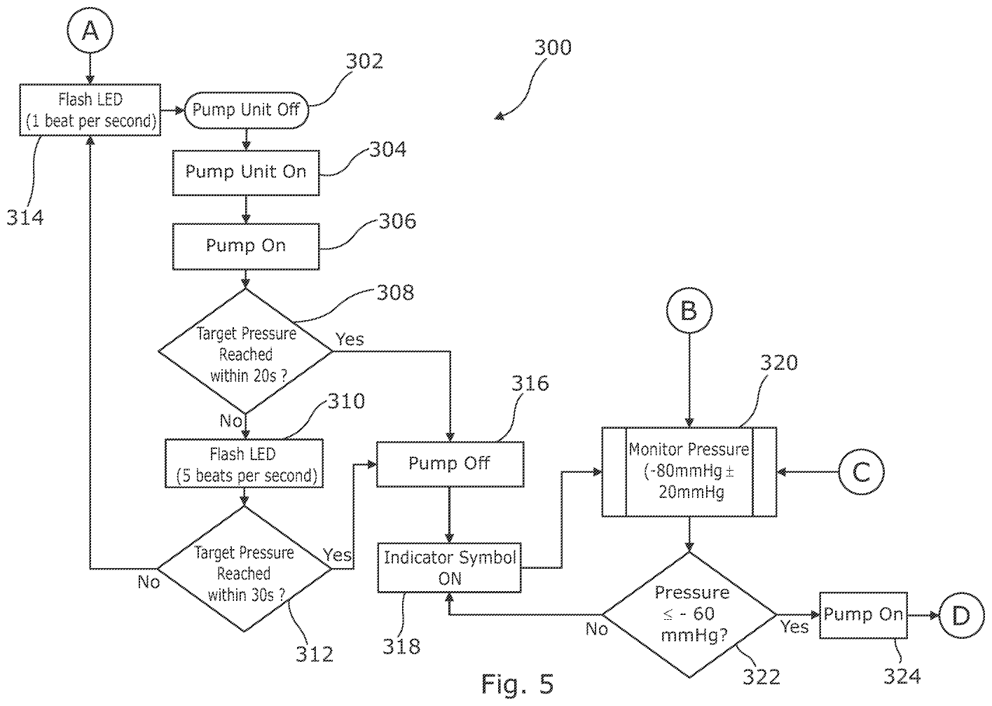

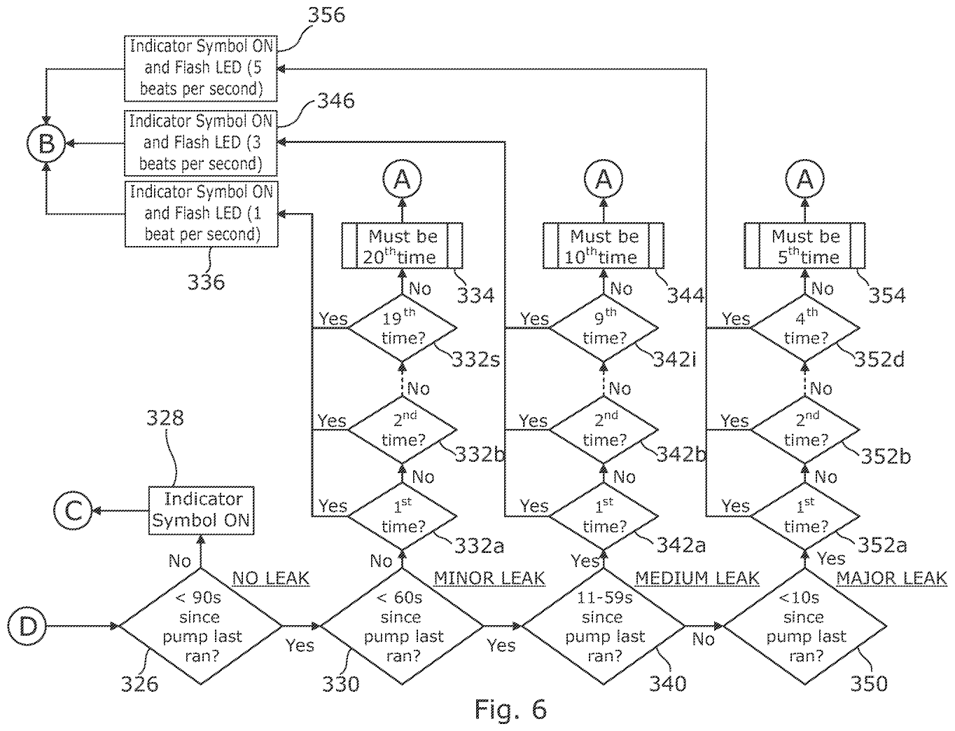

It will be appreciated that FIGS. 3 and 4 show a control system 100 and wound therapy apparatus 200, respectively, suitable for performing the method 10 shown in FIG. 1. However, it will further be appreciated that a similar arrangement may be used to perform the method 10′ of FIG. 2. Further, the control system 100 and wound therapy apparatus 200 may equally be operable to perform the method 300 of FIGS. 5 and 6 (discussed hereinbelow).

For instance, the processor 104 may be operable to receive an input signal 114 comprising data indicative of a time value corresponding to the time between successive operating cycles of the pump assembly 204 of the wound therapy apparatus 200. In such instances, the processor 104 may be configured to receive the input signal directly from a sensor (e.g. pressure sensor 215 or another sensor associated with the pump assembly 204) monitoring operation of the pump assembly 204. Alternatively, for instance where the control system 100 is operable to control operation of the pump assembly 204, the time value may be measured directly via the processor 104.

In this alternative arrangement, the processor 104 may be configured to analyse the input signal 114 (or use the directly measured time value) to determine a rate of change of pressure within the wound dressing. Specifically, the processor 104 is configured to determine the time between the end of a first operating cycle of the pump assembly 204 and the start of a second operating cycle of the pump assembly 204, and compare this time value with a second time value corresponding to the length of time of an operating cycle of the pump assembly 204 to determine a rate of change of pressure in the applied wound dressing 202. The rate of change of pressure is indicative of a leak level from the wound dressing 202, so can be used to determine the significance or severity of any leaks present.

With the determined rate of change of pressure, the processor 104 is configured to compare the determined rate of change of pressure to a plurality of leak thresholds as described herein to categorise the leak level associated with the determined rate of change of pressure into one of a plurality of leak level categories, and is configured to control operation of the wound therapy apparatus 200 in accordance with that categorisation as described herein.

FIGS. 5 and 6 illustrate a further embodiment of a method 300 in accordance with the invention.

The pump unit (e.g. the pump assembly 204) is initially provided in an “off” state (step 302). A user may activate the pump unit, for example, by pressing (and optionally holding) a button on the pump unit (step 304). The pump (e.g. pump 212) of the pump unit subsequently activates at step 306, here to reduce pressure within the applied wound dressing.

At step 308, a check is performed to determine whether the target pressure has been reached within a time period of 20 s. This is performed using a pressure sensor (e.g. sensor 215) associated with the wound dressing. The target pressure may be any suitable pressure value, but in the illustrated embodiment is set at −80 mmHg. If the target pressure is not reached within 20 s an output is provided in the form of a flashing first (e.g. red/amber) LED on the pump unit (step 310). The first LED may be one of indicators 214a, 214b, 214c of pump assembly 204, for example. Here, the first LED is flashed at a frequency of 5 beats per second to inform the user that the pump unit has been unable to achieve the target pressure. At step 312, a further check is performed, this time to determine whether the target pressure has been reached within a time period of 30 s. If the target pressure is not reached within 30 s the first LED is flashed at a frequency of 1 beat per second (step 314) before the pump unit is then switched off (step 302). This scenario may correspond to where a leak from the wound dressing is so great, or one or more components of the pump unit have malfunctioned, for example, such that the pump unit cannot reach the desired pressure level.

If either of the checks at step 308 or 312 return a positive response—i.e. the target pressure level has been reached within the wound dressing, the method moves on to step 316 where the pump of the pump unit is turned off, and at step 318 an indicator symbol (e.g. one of indicators 214a, 214b, 214c of pump assembly 204) on the pump unit is turned on to indicate to a user that the target pressure level has been reached. The indicator symbol preferably comprises a second LED on the pump unit—e.g. a green LED which may be permanently illuminated when the pressure level inside the wound dressing is acceptable and the pump of the pump unit is off.

Step 320 begins the pressure monitoring process of method 300. Specifically, the pressure within the wound dressing is monitored to check it is at the target pressure level (−80 mmHg), or within a predetermined range of the target pressure level (±20 mmHg). A check is performed at step 322 to determine whether the pressure within the wound dressing has dropped below −60 mmHg. If not, the method 300 loops back to step 318 where the indicator symbol is kept “on” before returning to step 320.

If the pressure within the wound dressing has dropped below −60 mmHg, the method 300 moves to step 324 where the pump of the pump unit is turned on. The method then proceeds through a series of steps to categorise the leak level from the wound dressing and take appropriate action. As is described herein, method 300 comprises measuring a time value indicative of the rate of change of pressure within the applied wound dressing. Specifically, the method 300 comprises measuring the time between successive operations of the pump unit to determine and subsequently categorise the leak level from the wound dressing.

At step 326, a check is performed to determine whether it has been less than 90 s between operations of the pump. If not, it is inferred that the pressure within the wound dressing has stayed within an acceptable range for longer than 90 s and the leak level is thus categorised in a no (or “acceptable”) leak category. At step 328, the indicator symbol is turned on (or kept on) to indicate an acceptable or no leak scenario.

If it has been less than 90 s since the previous operation of the pump unit, the method 300 moves to step 330 where a further check is performed to determine whether it has been less than 60 s between operations of the pump 212. If not, it is inferred that the pressure within the wound dressing has stayed within an acceptable range for between 60-90 s and the leak level is thus categorised in a minor leak category. The method 300 then comprises a series of steps (332a, 332b, 332s) to determine the number of times the determined leak level has been categorised as a minor leak. If this number is 19 or less, the method moves to step 336, wherein the indicator symbol is turned on (or kept on), but the first LED is flashed, here at a frequency of 1 beat per second. This indicates to the user that the pump unit is taking action to maintain the pressure level within the wound dressing, but there is a minor leak present. The user may then take steps to address the leak. Following this, the method moves back to steps 320, 322 etc. to repeat the pressure checks. If the determined leak level has been categorised as a minor leak 20 times (step 334), the method moves back to 314 where the first (red/amber) LED is flashed and the pump unit turned off to prevent excessive wear and energy usage of the pump. The second (green) LED would also be turned off in this instance.

Moving back to step 330, if it has been less than 60 s since the previous operation of the pump, the method 300 moves to step 340 where a further check is performed to determine whether it has been between 11-59 s since the previous operation of the pump. If so, it is inferred that the pressure within the wound dressing has stayed within an acceptable range for between 11-59 s and the leak level is thus categorised in a medium leak category. The method 300 then comprises a series of steps (342a, 342b, 342i) to determine the number of times the determined leak level has been categorised as a medium leak. If this number is 9 or less, the method moves to step 346, wherein the indicator symbol is turned on (or kept on), but the first LED is flashed, here at a frequency of 3 beats per second. This indicates to the user that the pump unit is taking action to maintain the pressure level within the wound dressing, but there is a medium leak present. This should prompt the user to take necessary steps to address the leak more urgently. Following this, the method moves back to steps 320, 322 etc. to repeat the pressure checks. If the determined leak level has been categorised as a medium leak 10 times (step 344), the method moves back to 314 where the first LED is flashed and the pump unit turned off to prevent excessive wear and energy usage of the pump. Again, the second (green) LED would also be turned off in this instance.

At step 350, a further check is performed to determine whether it has been less than 10 s since the previous operation of the pump. The outcome of this decision should always be “yes” given the checks performed at steps 326, 330 and 340. On the basis that is has been less than 10 s, it is inferred that the pressure within the wound dressing has only stayed within an acceptable range for less than 10 s and the leak level is thus categorised in a major leak category. The method 300 then comprises a series of steps (352a, 352b, . . . , 352d) to determine the number of times the determined leak level has been categorised as a major leak. If this number is 4 or less, the method moves to step 346, wherein the indicator symbol is turned on (or kept on), but the first LED is flashed, here at a frequency of 5 beats per second. This indicates to the user that the pump unit is taking action to maintain the pressure level within the wound dressing, but there is a major leak present that must be addressed as a matter of urgency. Following this, the method moves back to steps 320, 322 etc. to repeat the pressure checks. If the determined leak level has been categorised as a major leak 5 times (step 354), the method moves back to 314 where the first LED is flashed and the pump unit turned off to prevent excessive wear (e.g. of pump 212) and energy usage (e.g. of pump assembly 204).

In this way, method 300 controls the number of times the pump can be turned on (and hence the number of times the pump unit can operate) depending on the categorised leak level. This ensures that operation of the pump unit is limited more strongly for higher leak levels compared with lower leak levels. The different levels/frequencies at which the first LED is flashed dependent on leak level category is also designed to bring the leak level to the attention of the user—e.g. by flashing at a faster rate for higher leak levels. In an ideal scenario, a user would see the flashing indicator and take appropriate action to limit the leak from the wound dressing. If this is done in time—i.e. if this is done before 20 successive operations of the pump unit for a minor leak, before 10 successive operations of the pump unit for a medium leak, or before 5 successive operations of the pump unit for a major leak—the wound therapy apparatus may, as per method 300, continue to operate to provide the appropriate therapy. If, however, no appropriate action is taken to correct any leak, the method 300 ensures that the pump unit is prevented from operating so as to control the wear on, energy consumption of and noise generated by the wound therapy apparatus.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Each of the documents referred to above is incorporated herein by reference. Except in Examples, or where otherwise explicitly indicated, all numerical quantities in this description specifying amounts of materials, device dimension, and the like, are to be understood as modified by the word “about.”

Unless otherwise indicated, each chemical or composition referred to herein should be interpreted as being a commercial grade material which may contain the isomers, by-products, derivatives, and other such materials which are normally understood to be present in the commercial grade.

The one or more embodiments are described above by way of example only. Many variations are possible without departing from the scope of protection afforded by the appended claims.

1. A method for monitoring operation of a pressure gradient wound therapy apparatus, the method comprising:
measuring a parameter indicative of a rate of change of pressure within the applied wound dressing;
determining a rate of change of pressure within the applied wound dressing in dependence on the measured parameter, the rate of change of pressure corresponding to a leak level;
comparing the determined rate of change of pressure to a plurality of leak thresholds to categorise the leak level into one of a plurality of leak level categories; and
controlling the wound therapy apparatus in accordance with one or more predetermined actions in dependence on the leak level category.

2. The method as claimed in claim 1, comprising categorising the leak level into leak level categories corresponding to one or more of: a no leak category; a minor leak category; and a major leak category.

3. The method as claimed in claim 2, wherein one or more further leak level categories, including a medium or intermediate leak level category between a minor leak category and a major leak category.

4. The method as claimed in claim 1, comprising controlling an operating level of a component of the wound therapy apparatus in dependence on the leak level category.

5. The method as claimed in claim 4, wherein the component comprises a pump assembly of the wound therapy apparatus.

6. The method as claimed in claim 5, wherein, the operating level comprises a power output or motor speed of the pump assembly.

7. The method as claimed in claim 1, comprising outputting an indication to a user of the apparatus in dependence on the leak level category.

8. The method as claimed in claim 7, wherein the indication includes one or more of illuminating a light, controlling an associated display, and activating an alert to inform the user of the apparatus of the presence of a leak.

9. The method as claimed in claim 1, comprising controlling the wound therapy apparatus in accordance with one or more different instructions in dependence on the leak level category.

10. The method as claimed in claim 9, comprising:
controlling the wound therapy apparatus in accordance with a first set of instructions in dependence on a determination of a leak level in a first leak level category; and
controlling the wound therapy apparatus in accordance with a second set of instructions in dependence on a determination of a leak level in a second leak level category.

11. The method as claimed in claim 10, comprising controlling the wound therapy apparatus in accordance with a third set of instructions in dependence on a determination of a leak level in a third leak level category.

12. The method as claimed in claim 11, comprising controlling the wound therapy apparatus in accordance with a fourth set of instructions in dependence on a determination of a leak level in a fourth leak level category.

13. The method as claimed in claim 12, comprising:
taking no action in dependence on a determination of a leak level in a no leak category;
outputting an indication to a user of the apparatus in dependence on a determination of a leak level in a minor leak level category;
controlling an operating level of a pump assembly of the wound therapy apparatus in dependence on a determination of a leak level in a medium leak level category; and
preventing operation of the pump assembly in dependence on a determination of a leak level in a major leak level category.

14. The method as claimed in claim 1, comprising:
comparing the rate of change of pressure to a first leak threshold and categorising the corresponding leak level in a first leak level category if the rate of change of pressure is below the first leak threshold; and
categorising the corresponding leak level in a second leak level category if the rate of change of pressure is above the first leak threshold.

15. The method as claimed in claim 14, comprising:
comparing the rate of change of pressure to a second leak threshold and categorising the corresponding leak level in a second leak level category if the rate of change of pressure is below the second leak threshold but above the first leak threshold;
comparing the rate of change of pressure to a third leak threshold and categorising the corresponding leak level in a third leak level category if the rate of change of pressure is below the third leak threshold but above the second leak threshold; and
categorising the corresponding leak level in a fourth leak level category if the rate of change of pressure is above the third leak threshold.

16. The method as claimed in claim 1, comprising comparing the determined rate of change of pressure with a rate of change of pressure observed at one or more preceding time periods.

17. The method as claimed in claim 16, comprising obtaining an average rate of change of pressure observed at a plurality of preceding time periods, and comparing the determined rate of change of pressure with the average rate of change of pressure observed at the plurality of preceding time periods.

18. The method as claimed in claim 1, wherein the parameter indicative of the rate of change of pressure within the applied wound dressing comprises a pressure value, and the method comprises:
obtaining a first pressure value corresponding to the pressure within an applied wound dressing within a first time period;
comparing the first pressure value with a second pressure value corresponding to the pressure within the applied wound dressing within a preceding time period to determine the rate of change of pressure within the applied wound dressing.

19. The method as claimed in claim 18, comprising obtaining a plurality of pressure values within the first time period, and obtaining an average of the plurality of pressure values obtained within the first time period to obtain the first pressure value.

20. The method as claimed in claim 18, comprising determining a rate of change of pressure within the wound dressing as follows:
$$Rate of change of pressure = \vert \frac{{P}_{m} - {P}_{m - 1}}{{T}_{m} - {T}_{m - 1}} \vert$$
where Pₘ is the first pressure value, Pₘ₋₁ is the second pressure value, Tₘ corresponds to a time stamp of the first time period, and Tₘ₋₁ corresponds to a time stamp of the preceding time period.

21. The method as claimed in claim 1, comprising measuring the or each parameter indicative of a rate of change of pressure only when a pump assembly of the wound therapy apparatus is inactive.

22. The method as claimed in claim 1, wherein the parameter indicative of the rate of change of pressure within the applied wound dressing comprises, or is calculated from, a time value; and wherein the time value corresponds to the time between successive operating cycles of a pump assembly of the wound therapy apparatus.

23. The method as claimed in claim 22, comprising:
measuring a time value corresponding to the time between the end of a first operating cycle of the pump assembly and the start of a second operating cycle of the pump assembly; and
comparing the time value with a second time value corresponding to the length of time of an operating cycle of the pump assembly to determine a ratio of the time for which the pump assembly is not operating and the time for which the pump assembly is operating.

24. A control system control system for monitoring operation of a pressure gradient wound therapy apparatus, the control system comprising one or more controllers, the control system being configured to:
receive an input signal indicative of a parameter indicative of a rate of change of pressure within the applied wound dressing;
determine a rate of change of pressure within the applied wound dressing in dependence on the parameter, the rate of change of pressure corresponding to a leak level;
compare the determined rate of change of pressure to a plurality of leak thresholds to categorise the leak level into one of a plurality of leak level categories; and
output a control signal to control the wound therapy apparatus in accordance with one or more predetermined actions in dependence on the leak level category.

25. The wound therapy apparatus of claim 24, comprising a negative pressure wound therapy apparatus.
